(19) United States
(12) Patent Application Publication
(10) Pub. No.: US 2009/0299472 A1
(43) Pub. Date: Dec. 3, 2009

(54) LIGHTWEIGHT BREAST PROSTHESIS

(76) Inventor: Chun-Kai HUANG, Huatan Township (TW)

Correspondence Address: Muncy, Geissler, Olds & Lowe, PLLC, P.O. BOX 1364, FAIRFAX, VA, 22038-1364, US

(21) Appl. No.: 12/418,742

(22) Filed: Apr. 6, 2009

(30) Foreign Application Priority Data
May 27, 2008 (TW) 097209244

(51) Int. Cl. A61F 2/52

(52) U.S. Cl. 623/7

(57) ABSTRACT

A lightweight breast prosthesis is formed as an enclosed body having therein at least two compartments chamber, wherein one of the compartments chamber is filled with silicone gel, and the other compartment chamber or compartments chamber are filled with a substance lighter than silicone gel. Thus, the breast prosthesis, which is soft and tender like silicone gel but lighter than a breast prosthesis made entirely of silicone gel, can serve either as a complete prosthetic breast or as a breast shape-supplementing pad for locally supplementing the breast shape or making up a partially removed breast.

### BACKGROUND OF THE INVENTION

1. Technical Field

The present invention relates to breast prostheses and, more particularly, to a breast prosthesis that, when touched, provides the softness and tenderness of silicone gel but is lighter than a breast prosthesis made entirely of silicone gel so as to reduce the otherwise heavy burden on a user while serving either as a complete prosthetic breast or as a breast shape-supplementing pad for locally supplementing the breast shape or making up a partially removed breast.

2. Description of Related Art

For those who have undergone mastectomy because of breast cancer, prosthetic breasts or breast pads are useful not only in breast shape reconstruction but also in restoring the patients' self-confidence. Conventional prosthetic breasts or breast pads are filled entirely with silicone gel to simulate the natural feel of human muscles. However, due to their considerable weight, these prosthetic breasts or breast pads have also become a burden on the wearers. In particular, prosthetic breasts made of silicone gel tend to apply an excessively heavy and long-lasting load on the wearers' chests and therefore lead to bad body postures such as hunchbackedness or even scoliosis.

### BRIEF SUMMARY OF THE INVENTION

In view of the aforementioned shortcomings of the existing prosthetic breasts or local supplementary breast pads made entirely of silicone gel, the present invention aims to provide a lightweight breast prosthesis that is as tender and comfortable to the touch as silicone gel but overcomes the drawbacks of the existing prosthetic breasts or local supplementary breast pads.

A breast prosthesis having the aforesaid desired features is formed as an enclosed body wrapped by a flexible film. The enclosed body includes therein at least two isolated compartments chamber, wherein at least one of the compartments chamber is filled with silicone gel, and at least another one of the compartments chamber is filled with a substance lighter than silicone gel. Therefore, the enclosed body including the silicone gel-filled compartment chamber and the compartment chamber filled with the substance lighter than silicone gel has a lighter overall weight than a conventional all-silicone gel prosthetic breast or local supplementary breast pad. In addition, the silicone gel-filled compartment chamber according to the present invention provides the same softness and tenderness as the conventional silicone gel-made prosthetic breast or local supplementary breast pad.

In addition to including two isolated compartment chambers, the breast prosthesis having the foregoing desired features has another preferred embodiment formed as an enclosed body including three compartment chambers, wherein at least one of the compartment chamber is filled with a substance lighter than silicone gel so that, when used as a prosthetic breast or a local supplementary breast pad, the three-compartment chamber enclosed body not only is lighter than an all-silicone gel prosthetic breast or local supplementary breast pad, but also forms an elastic body having a fixed shape after the silicone gel is set. This configuration allows the breast prosthesis to maintain its designed shape as a breast pad and have a much lighter weight than a prior art counterpart, thereby reducing the burden put on the user.

In the aforesaid breast prosthesis, the flexible film is a thermoplastic polyurethane (TPU) film or any other film available and well known in the art. The shape of the enclosed body wrapped by the film can be any other fixed shape of an elastic body designed for modifying the breast shape than those of the conventional prosthetic breasts or local supplementary breast pads.

In the aforesaid breast prosthesis, the substance that is lighter than silicone gel and used to fill the compartment chamber(s) can be air, a liquid, or a foamed material, or other light density materials that is lighter than silicone gel.

In the aforesaid breast prosthesis, the enclosed body may have its outer surface partially or entirely covered by a fabric for separating the plastic film so that the breast prosthesis is suitable for users who have allergy-prone body constitutions or sensitive skin or users who are advised to avoid direct contact with plastic products.

### BRIEF DESCRIPTION OF THE SEVERAL VIEWS OF THE DRAWINGS

The above and other objectives of the present invention as well as its configuration, features, and functions will be best understood by reference to the following detailed description of illustrative embodiments in conjunction with the accompanying drawings, wherein:

FIG. 1 is a sectional view of a breast prosthesis according to a first preferred embodiment of the present invention as a prosthetic breast;

FIG. 2 is a sectional view of a breast prosthesis according to a second preferred embodiment of the present invention as a prosthetic breast;

FIG. 3 is a sectional view of a breast prosthesis according to a third preferred embodiment of the present invention as a local supplementary breast pad; and

FIG. 4 is a sectional view of a breast prosthesis according to a fourth preferred embodiment of the present invention as a local supplementary breast pad.

### DETAILED DESCRIPTION OF THE INVENTION

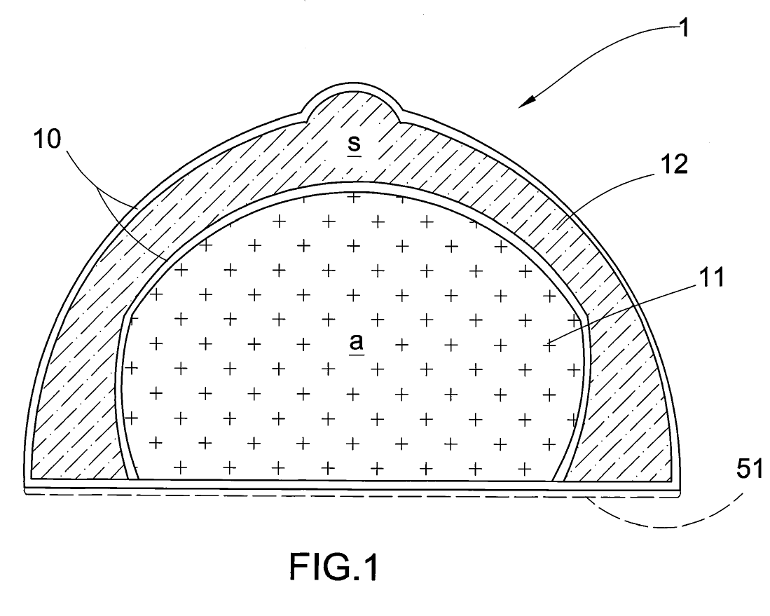

Referring to FIG. 1, a breast prosthesis according to a first preferred embodiment of the present invention includes a breast-shape enclosed body 1. The enclosed body 1 is wrapped by a film 10 and internally partitioned into two compartments chamber 11, 12 also by the film 10. The compartment chamber 12 forms an outer compartment chamber along the contour of the enclosed body 1. Meanwhile, the compartment chamber 11 is surrounded by the outer compartment chamber 12 and thus forms a relatively inner compartment chamber. In the present embodiment, the outer compartment chamber 12 is preferably filled with silicone gel s while the inner compartment chamber 11 is filled with a substance a lighter than silicone gel, or vice versa. In the present embodiment, the substance lighter than silicone gel can be selected from a variety of substances such as air, a liquid, or a foamed material, or other light density materials that is lighter than silicone gel. In the enclosed body 1 according to the first preferred embodiment, the air-filled inner compartment chamber 11 is partially surrounded by the silicone gel-filled outer compartment chamber 12. Therefore, while the silicone gel-filled compartment chamber 12 provides the same softness and shape as the conventional all-silicone gel prosthetic breast, the compartment chamber 11 filled with the light-than-silicone gel substance contributes to a lighter overall weight of the enclosed body 1 than the conventional prosthetic breast and helps maintain the outer shape of the enclosed body 1.

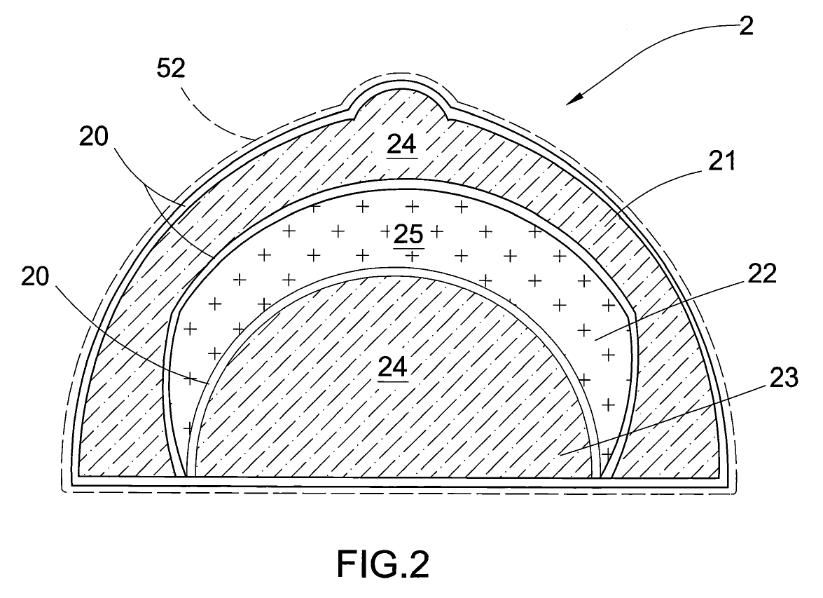

FIG. 2 illustrates a breast prosthesis according to a second preferred embodiment of the present invention, wherein the breast prosthesis also includes a breast-shaped enclosed body 2. The enclosed body 2 is wrapped by a film 20 and internally partitioned by the film 20 into three compartments chamber 21, 22, 23, wherein the first compartment chamber 21 is formed along the contour of the enclosed body 2, and the second compartment chamber 22 and the third compartment chamber 23 are sequentially arranged below the first compartment chamber 21. In the second preferred embodiment, the first and the third compartments chamber 21, 23 are filled with silicone gel 24 while the second compartment chamber 22 is filled with a substance 25 lighter than silicone gel, or vice versa. In the second preferred embodiment, the first and the third compartments chamber 21, 23 are filled with silicone gel 24 to give the entire outer layer of the enclosed body 2 a feel of silicone gel. However, the second compartment chamber 22 that is located between the first and the third compartments chamber 21, 23 and filled with the substance 25 lighter than silicone gel makes the enclosed body 2 lighter than the conventional all-silicone gel prosthetic breast and helps maintain the outer shape of the enclosed body 2.

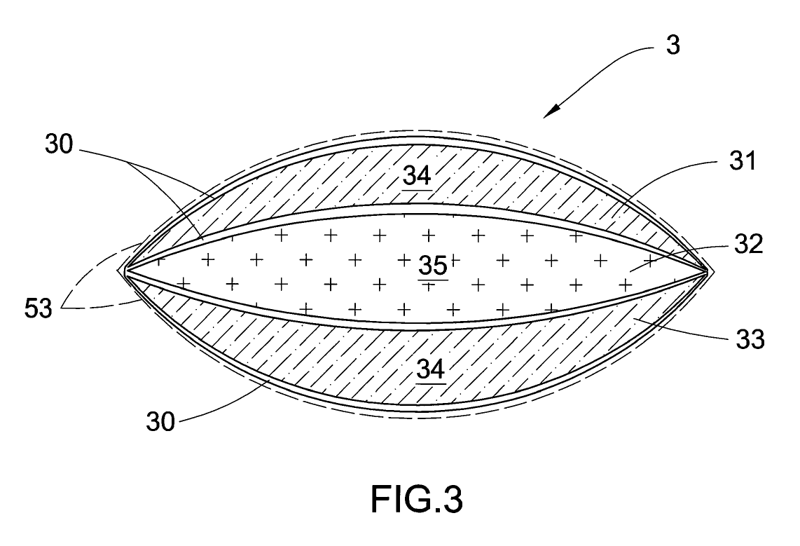

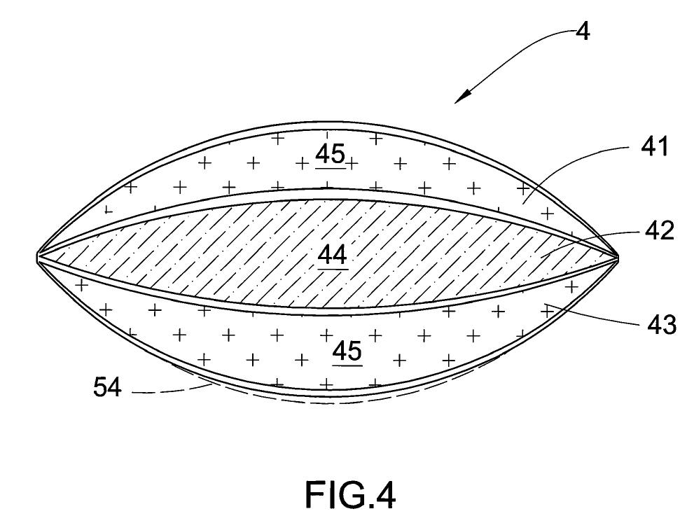

Referring to FIG. 3 for a breast prosthesis according to a third preferred embodiment of the present invention, the breast prosthesis includes an enclosed body 3 formed as a fixed shape-pad for making up a partially removed breast or locally supplementing the breast shape rather than replacing an entirely removed breast. The enclosed body 3 is wrapped by a film 30 and internally partitioned into three compartments chamber 31, 32, 33 by the film 30. The first compartment chamber 31 and the third compartment chamber 33 are formed along the contour of the enclosed body 2 while the second compartment chamber 32 is interposed between the first and the third compartments chamber 31, 33. In the third preferred embodiment, the first and the third compartments chamber 31, 33 are filled with silicone gel 34 while the second compartment chamber 32 is filled with a substance 35 lighter than silicone gel, or vice versa, as shown in FIG. 4 which illustrates an enclosed body 4 according to a fourth preferred embodiment of the present invention. In the enclosed body 4, a first compartment chamber 41 and a third compartment chamber 43 are filled with a substance 45 lighter than silicon gel while a second compartment chamber 42 is filled with silicone gel 44. The enclosed body 3 in the third preferred embodiment has outer layers that feel like silicone gel when touched. Also, the enclosed body 3 is lighter than the conventional all-silicone gel prosthetic breast and has an appropriate shape for completing the breast shape. In comparison, the outer layers of the enclosed body 4 in the fourth preferred embodiment are filled with the substance 45 lighter than silicone gel and therefore lack the feel of silicone gel. However, the enclosed body 4 is even lighter than the enclosed body 3 in the third preferred embodiment.

The lightweight breast prosthesis formed by any of the aforesaid enclosed bodies can be additionally provided with a surface fabric that covers an outer surface of the enclosed body so as to cater for the need of those who have allergy-prone body constitutions or sensitive skin or those who are advised to avoid direct contact with plastic products. The surface fabric may cover the entire outer surface of the enclosed body, as demonstrated by surface fabrics 52, 53 in FIGS. 2, 3, or be attached to only a predetermined or partial area of the outer surface of the enclosed body, as demonstrated by surface fabrics 51, 54 in FIGS. 1, 4.

The preferred embodiments disclosed herein are intended to illustrate implementation of the objectives of the present invention but not to limit the structural features of the present invention. Changes or modifications can be made to the disclosed embodiments by a person of ordinary skill in the art without departing from the concept of the present invention. Therefore, the scope of the present invention is defined only by the appended claims.

1. A lightweight breast prosthesis, comprising:
an enclosed body wrapped by a flexible film and comprising therein at least two isolated compartments chamber, wherein at least one said compartment chamber is filled with silicone gel, and at least another said compartment chamber is filled with a substance lighter than silicone gel.

2. The lightweight breast prosthesis of claim 1, wherein the enclosed body comprises three said isolated compartments chamber, one or two said compartments chamber being filled with silicone gel while the other two or one said compartment chamber being filled with the substance lighter than silicone gel.

3. The lightweight breast prosthesis of claim 2, wherein one of the three isolated compartments chamber in the enclosed body is filled with silicone gel and interposed between the other two compartments chamber filled with the substance lighter than silicone gel.

4. The lightweight breast prosthesis of claim 2, wherein one of the three isolated compartments chamber in the enclosed body is filled with the substance lighter than silicone gel and interposed between the other two compartments filled with silicone gel.

5. The lightweight breast prosthesis of claim 1, wherein the substance lighter than silicone gel is one of air, a liquid, and a foamed material.

6. The lightweight breast prosthesis of claim 1, wherein the enclosed body is breast-shaped.

7. The lightweight breast prosthesis of claim 1, wherein the enclosed body is a pad of any shape designed for locally supplementing a breast shape.

8. The lightweight breast prosthesis of claim 1, wherein the flexible film is a thermoplastic polyurethane (TPU) film.

9. The lightweight breast prosthesis of claim 1, further comprising a surface fabric covering an entire outer surface of the enclosed body.

10. The lightweight breast prosthesis of claim 1, further comprising a surface fabric covering a predetermined area of an outer surface of the enclosed body.
